(19) United States
(12) Patent Application Publication
(10) Pub. No.: US 2008/0261995 A1
(43) Pub. Date: Oct. 23, 2008

(54) Pharmaceutical Combination of a Pde-5 Inhibitor and a 5-Alpha Reductase Inhibitor

(75) Inventors: Carl Erik Johan Mastrell, New York, NY (US); Michael Allen Suesserman, New York, NY (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 12/092,287

(22) PCT Filed: Dec. 13, 2006
(86) PCT No.: PCT/IB2006/003649

§ 371 Date: May 1, 2008

Related U.S. Application Data
(60) Provisional application No. 60/752,805, filed on Dec. 21, 2005.
(60) Provisional application No. 60/757,747, filed on Jan. 9, 2006.

(51) Int. Cl. A61K 31/473; A61K 31/519; A61K 31/496; A61P 13/00

(52) U.S. Cl. 514/253.09; 514/284; 514/262.1

(57) ABSTRACT

This invention relates to the combined use of a PDE5 inhibitor and a 5-alpha reductase antagonist in the treatment of lower urinary tract symptoms (LUTS), such as urgency, frequency, nocturia and urge incontinence.

This application claims priority to U.S. Provisional Application Ser. Nos. 60/752,805 and 60/757,747, filed Dec. 21, 2005 and Jan. 9, 2006, respectively.

This invention relates to the combined use of a PbE5 inhibitor and a 5-alpha reductase inhibitor in the treatment of lower urinary tract symptoms (LUTS).

LUTS comprise three groups of urinary symptoms, which may be defined as storage (irritative), voiding (obstructive) and post-micturition symptoms. Storage symptoms comprise urgency, frequency, nocturia, urgency incontinence and stress incontinence, which can be associated with overactive bladder (OAB) and benign prostatic hyperplasia (BPH). Voiding symptoms comprise hesitancy, poor flow, intermittency, straining and dysuria. Post-micturition symptoms comprise terminal dribbling, post-void dribbling, and a sense of incomplete emptying.

Over Active Bladder (OAB) is defined as urgency, with or without urge incontinence, usually with frequency and nocturia [Abrams, et al., Neurourology and Urodynamics 21:167-178 (2002)]. Prevalence of OAB in men and women is similar, with approximately 16% of the population of the USA suffering from the condition [Stewart et al, Prevalence of Overactive Bladder in the United States: Results from the NOBLE Program; Abstract Presented at the 2nd International Consultation on Incontinence, July 2001, Paris, France].

The terms OAB Wet and OAB Dry describe OAB patients with or without urinary incontinence respectively. Until recently, the cardinal symptom of OAB was believed to be urinary incontinence. However, with the advent of the new terms this is clearly not meaningful for the large number of sufferers who are not incontinent (i.e., OAB Dry patients). Thus, a recent study from Liberman et al [‘Health Related Quality of Life Among Adults with Symptoms of Overactive Bladder: Results From A US Community-Based Survey’; Urology 57(6), 1044-1050, 2001] examined the impact of all OAB symptoms on the quality of life of a community-based sample of the US population. This study demonstrated that individuals suffering from OAB without any demonstrable loss of urine have an impaired quality of life when compared with controls.

BPH is a chronically progressive disease that can lead to complications such as acute urinary retention, recurrent urinary tract infections, bladder stones and renal dysfunction. The prevalence and average severity of LUTS associated with BPH in men increases with age.

BPH leads to an increase in prostate volume, creating urethral and bladder outflow obstruction as well as secondary changes in bladder function. The effects of this are manifested by both storage (irritative) and voiding (obstructive) symptoms.

WO 2005/018620 discloses the use of a nitric oxide synthase (NOS) cofactor in the treatment of sexual dysfunction. A preferred cofactor is tetrahydrobiopterin (BH₄), or a precursor or analogue thereof. Such compounds are distinguished over PDE5 inhibitors in the document (see page 5 lines 11-12). When the sexual dysfunction is associated with BPH, the document teaches that administration of the NOS cofactor may be combined with a 5-alpha reductase inhibitor.

U.S. Pat. No. 6,642,274 discloses a method of treating prostate disorders comprising administering various medicaments directly to the mucosal membranes of the lower urinary tract. Seven classes of therapeutic compounds are suggested for use in the method, including phosphodiesterase inhibitors and anti-androgens (including the 5-alpha reductase inhibitor, finasteride). There is also a suggestion that such compounds can be used alone or in combination in the disclosed method, but there is no explicit mention of a combination of a PDE5 inhibitor and a 5-alpha reductase inhibitor.

US 2004/0180958 (and its equivalent WO 2004/054560) discloses the use of alpha-2-delta ligands in the treatment of LUTS, other than urinary incontinence, associated with OAB and/or BPH. Their combined use with PDE5 inhibitors is also disclosed in the treatment of LUTS associated with OAB and/or BPH. The combined use of an alpha-2-delta ligand and a 5-alpha reductase inhibitor is also disclosed in the treatment of LUTS associated with BPH.

WO 99/65228 relates to the treatment of testosterone deficiency in men while simultaneously protecting the prostate. The combinations contain a natural or synthetic androgen; and a compound selected from various classes of compound including testosterone 5-alpha reductase inhibitors and phosphodiesterase inhibitors.

WO 99/02161 discloses the use of selective inhibitors of PDE1, PDE4 and PDE5 in the treatment of prostatic diseases.

EP 1020190 discloses the use of PDE5 inhibitors in the treatment of BPH and their combination with α-antagonists for this purpose.

WO 01/17480 discloses the treatment of urinary disorders in a mammal comprising administering therapeutic compounds directly to the mucosal membranes of the lower urinary tract. Preferred groups of compounds are stated to be autocoids, cytokines, chemotherapeutic agents, alpha-receptor antagonists, prostaglandin dehydrogenase inhibitors, phosphodiesterase inhibitors, anticholinergic and antispasmodic agents.

WO 2005/042741 discloses the use of 5-alpha reductase gene expression inhibitors in the treatment of BPH and incontinence, optionally in association with a PDE5 inhibitor.

WO 2006/108519 (published on 19 Oct. 2006, after the priority date of the present application) claims a pharmaceutical formulation or combination pack for the treatment of BPH comprising at least one 5-alpha reductase inhibitor and at least one PDE5 inhibitor in controlled release form or at least one PDE5 inhibitor with a long half-life.

WO 01/27112 and WO 01/27113 each disclose a series of pyrazolo[4,3-d]pyrimidin-7-ones which are PDE5 inhibitors. The compounds are indicated, amongst other things, in the treatment of BPH, bladder outlet obstruction and incontinence.

WO 99/58478 and its priority document EP 0957073 disclose derivatives of 3,3-diphenylpropylamines, including fesoterodine [R-(+)-isobutyric acid 2-(3-diisopropylamino-1-phenylpropyl)-4-hydroxymethylphenyl ester, see page 62 lines 15-16 of WO 99/58478]. The compounds are indicated in the treatment of urinary incontinence, amongst other things.

WO 89/06644 and its equivalent EP 325571 discloses a group of 3,3-diphenylpropylamines, including tolterodine [(+)-NN-diisopropyl-3-(2-hydroxy-5-methylphenyl)-3-phenylpropylamine, Example 22]. The compounds are indicated in the treatment of urinary incontinence.

WO 94/11337 discloses a group of 3,3-diphenylpropylamines, including (+)-N,N-diisopropyl-3-(2-hydroxy-5-hydroxymethylphenyl)-3-phenylpropylamine (Example 1), which is also formed by metabolism of tolterodine. The compounds are indicated in the treatment of urinary incontinence.

It has now been found that PDE5 inhibitors and 5-alpha reductase inhibitors are particularly useful when used together in the treatment of LUTS.

Thus, in accordance with a first aspect of the present invention there is provided a pharmaceutical formulation comprising a PDE5 inhibitor and a 5-alpha reductase inhibitor.

In accordance with a second aspect of the invention, there is provided the use of a PDE5 inhibitor and a 5-alpha reductase inhibitor in the manufacture of a medicament for the treatment of LUTS. There is also provided the use of a PDE5 inhibitor and a 5-alpha reductase inhibitor in the manufacture of a medicament for the treatment of LUTS other than the symptoms of BPH.

In accordance with a third aspect of the invention, there is provided a method of treatment of LUTS, comprising simultaneous, separate or sequential administration of a PDE5 inhibitor and a 5-alpha reductase inhibitor to a patient in need of such treatment. There is also provided a method of treatment of LUTS other than the symptoms of BPH, comprising simultaneous, separate or sequential administration of a PDE5 inhibitor and a 5-alpha reductase inhibitor to a patient in need of such treatment.

In accordance with a fourth aspect of the invention, there are provided pharmaceutical products comprising a PDE5 inhibitor and a 5-alpha reductase inhibitor as a combined preparation for simultaneous, separate or sequential use in the treatment of LUTS. There is also provided pharmaceutical products comprising a PDE5 inhibitor and a 5-alpha reductase inhibitor as a combined preparation for simultaneous, separate or sequential use in the treatment of LUTS other than the symptoms of BPH.

The various aspects of the invention are referred to together herein as “the combinations of the invention”.

The LUTS of greatest interest are urgency, frequency, nocturia and urge incontinence, especially urgency.

The combinations of the invention are suitable for treating both men and women, although LUTS associated with BPH will only be found in men. Men suffering from both LUTS and male erectile dysfunction (MED) may also gain relief from MED symptoms through receiving the combinations of the invention.

PDE5 inhibitors suitable for use in the invention include, but are not limited to:
(i) the PDE5 inhibitors mentioned in International Patent Application Nos. WO 03/000691; WO 02/64590; WO 02/28865; WO 02/28859; WO 02/38563; WO 02/36593; WO 02/28858; WO 02/00657; WO 02/00656; WO 02/10166; WO 02/00658; WO 01/94347; WO 01/94345; WO 00/15639 and WO 00/15228;
(ii) the PDE5 inhibitors mentioned in U.S. Pat. Nos. 6,143,746; 6,143,747 and 6,043,252;
(iii) the pyrazolo[4,3-d]pyrimidin-7-ones disclosed in EP 0463756; the pyrazolo[4,3-d]pyrimidin-7-ones disclosed in EP 0526004; the pyrazolo[4,3-d]pyrimidin-7-ones disclosed in WO 93/06104; the isomeric pyrazolo[3,4-d]pyrimidin-4-ones disclosed in WO 93/07149; the quinazolin-4-ones disclosed in WO 93/12095; the pyrido[3,2-d]pyrimidin-4-ones disclosed in WO 94/05661; the purin-6-ones disclosed in WO 94/00453; the pyrazolo[4,3-d]pyrimidin-7-ones disclosed in WO 98/49166; the pyrazolo[4,3-d]pyrimidin-7-ones disclosed in WO 99/54333; the pyrazolo[4,3-d]pyrimidin-4-ones disclosed in EP 0995751; the pyrazolo[4,3-d]pyrimidin-7-ones disclosed in WO 00/24745; the pyrazolo[4,3-d]pyrimidin-4-ones disclosed in EP 0995750; the hexahydropyrazino[2′,1′:6,1]pyrido[3,4-b]indole-1,4-diones disclosed in WO95/19978; the pyrazolo[4,3-d]pyrimidin-4-ones disclosed in WO 00/27848; the imidazo[5,1-f] [1,2,4]triazin-ones disclosed in EP 1092719 and WO 99/24433; the bicyclic compounds disclosed in WO 93/07124; the pyrazolo[4,3-d]pyrimidin-7-ones disclosed in WO 01/27112; the pyrazolo[4,3-d]pyrimidin-7-ones disclosed in WO 01/27113; the compounds disclosed in EP 1092718; the compounds disclosed in EP 1092719; the tricyclic compounds disclosed in EP 1241170; the alkyl sulphone compounds disclosed in WO 02/074774; the compounds disclosed in WO 02/072586; the compounds disclosed in WO 02/079203; and the compounds disclosed in WO 02/074312;
(iv) 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (EP 0463756); 5-(2-ethoxy-5-morpholinoacetylphenyl)-1-methyl-3-n-propyl-1,6-dihydro-7H-pyrazolo[4,3-d]pyrimidin-7-one (EP 0526004); 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO 98/49166); 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (WO99/54333), (+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d]pyrimidin-7-one (WO 99/54333); 5-[2-ethoxy-5-(4-ethyl-piperazine-1-sulphonyl)-pyridin-3-yl]-3-ethyl-2-[2-methoxy-ethyl]-2,6-dihydro-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (WO 01/27113, Example 8) [the free base and besylate salt are of particular interest]; 5-[2-iso-butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (WO 01/27113, Example 15); 5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (WO 01/27113, Example 66); 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one (WO 01/27112, Example 124); 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one (WO 01/27112, Example 132); (6R,12aR)-2,3,6,7,12,12a-hexahydro-2-methyl-6-(3,4-methylenedioxyphenyl)pyrazino[2′,1′:6,1]pyrido[3,4-b]indole-1,4-dione (tadalafil, IC-351, Clalis®), i.e., Examples 78 and 95 of WO95/19978, as well as the compounds of Examples 1, 3, 7 and 8; 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil, LEVITRA®) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e., Examples 20, 19, 337 and 336 of WO99/24433; Example 11 of WO93/07124 (EISAI); compounds 3 and 14 from Rotella, D. P, J. Med. Chem., 2000, 43, 1257; 4-(4-chlorobenzyl)amino-6,7,8-trimethoxyquinazoline; N-[[3-(4,7-dihydro-1-methyl-7-oxo-3-propyl-1H-pyrazolo[4,3-d]-pyrimidin-5-yl)-4-propxyphenyl]sulfonyl]-1-methyl2-pyrrolidinepropanamide [“DA-8159” (Example 68 of WO00/27848)]; and 7,8-dihydro-8-oxo-6-[2-propoxyphenyl]-1H-imidazo[4,5-g]quinazoline and 1-[3-[1-[(4-fluorophenyl)methyl]-7,8-dihydro-8-oxo-1H-imidazo[4,5-g]quinazolin-6-yl]-4-propoxyphenyl]carboxamide;
(v) 4-bromo-5-(pyridylmethylamino)-6-[3-(4-chlorophenyl)-propoxy]-3(2H)pyridazinone; 1-[4-[(1,3-benzodioxol-5-ylmethyl)amiono]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furazlocillin; cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridylmethylamino)-6-(3-(4-chlorophenyl) propoxy)-3-(2H)pyridazinone; 1-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; WO 96/26940); Pharmaprojects No. 5069 (Schering Plough); GF-196960 (Glaxo Wellcome); E-8010 and E-4010 (Eisai); Bay-38-3045 & 38-9456 (Bayer); FR229934 and FR226807 (Fujisawa); and Sch-51866; and pharmaceutically acceptable salts and solvates thereof.

Preferred PDE5 inhibitors include: 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil), particularly sildenafil citrate; (6R,12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351 or tadalafil); 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil); 5-(5-acetyl-2-butoxy-3-pyridinyl)-3-ethyl-2-(1-ethyl-3-azetidinyl)-2,6-dihydro-7H-pyrazolo[4,3-o]pyrimidin-7-one; 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one; 5-[2-ethoxy-5-(4-ethyl-piperazine-1-sulphonyl)-pyridin-3-yl]-3-ethyl-2-[2-methoxy-ethyl]-2,6-dihydro-pyrazolo[4,3-d]pyrimidin-7-one; 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide (TA-1790); 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide (DA 8159); and pharmaceutically acceptable salts and solvates thereof.

More preferably, the PDE5 inhibitor is selected from sildenafil, tadalafil, vardenafil, DA-8159 and 5-[2-ethoxy-5-(4-ethyl-piperazine-1-sulphonyl)-pyridin-3-yl]-3-ethyl-2-[2-methoxy-ethyl]-2,6-dihydro-pyrazolo[4,3-d]pyrimidin-7-one, and pharmaceutically acceptable salts and solvates thereof.

Most preferably the PDE5 inhibitor is selected from sildenafil, 5-[2-ethoxy-5-(4-ethyl-piperazine-1-sulphonyl)-pyridin-3-yl]-3-ethyl-2-[2-methoxy-ethyl]-2,6-dihydro-pyrazolo[4,3-d]pyrimidin-7-one, and pharmaceutically acceptable salts and solvates thereof. Sildenafil citrate is a preferred salt of sildenafil. The besylate salt is a preferred salt of 5-[2-ethoxy-5-(4-ethyl-piperazine-1-sulphonyl)-pyridin-3-yl]-3-ethyl-2-[2-methoxy-ethyl]-2,6-dihydro-pyrazolo[4,3-d]pyrimidin-7-one.

Suitable 5-alpha reductase inhibitors include:

(a) finasteride, commercially available as Proscar®, EP 155196, having the structure

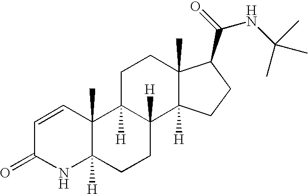

(b) dutasteride, commercially available as Avodart®, WO 95/07926, having the structure

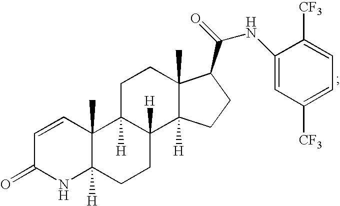

(c) izonsteride, EP 703221, having the structure

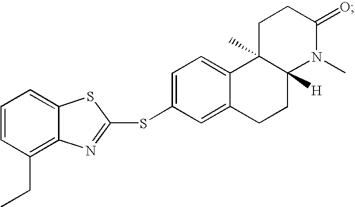

(d) idronoxil, U.S. Pat. No. 6,599,536, having the structure

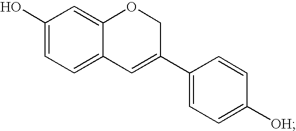

(e) epristeride, EP 289327, having the structure

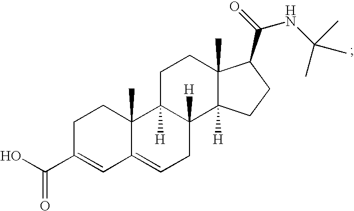

(f) serenoa repens, commercially available as Permixon®, a natural product which is the n-hexane lipidosterolic extract of the pulp and seed of the dwarf American palm, also known as Saw palmetto, see EP 68055;
(g) PHL 00801, commercially available as Prostatonin®, a natural product which is an extract of Pygeum africanum; and pharmaceutically acceptable salts and solvates thereof.

Especially preferred are: finasteride; dutasteride; and pharmaceutically acceptable salts and solvates thereof. Usually, these compounds are used in their free (i.e., non-salt) forms.

The combinations of the invention may have the advantage that the two components (i.e., the PDE5 inhibitor and the 5-alpha reductase inhibitor) act synergistically to produce an unexpectedly potent effect and/or an unexpectedly favourable level of side-effects in comparison with the corresponding total dosage of one of the components on its own. In addition, the combinations of the invention may have a longer duration of action, improved selectivity, or other more useful properties compared with the prior art.

The components of the combinations of the present invention are prepared by methods well known to those skilled in the art. Specifically, the patents, patent applications and publications, mentioned above, each of which is hereby incorporated by reference, exemplify components which can be used in combinations, pharmaceutical formulations, methods and kits in accordance with the present invention, and refer to methods of preparing those components.

The term ‘solvate’ is used herein to describe a molecular complex comprising a compound and one or more pharmaceutically acceptable solvent molecules, for example, ethanol. In a hydrate, the solvent is water.

Pharmaceutically acceptable salts of the components suitable for use in the invention include the acid addition and base salts thereof where the component compound has a basic centre or is ionizable, respectively. It will be apparent to those skilled in the art that not all components suitable for use in the invention will form salts readily. In addition, it will be apparent that serenoa repens and PHL 00801 are complex mixtures that may already contain salts and solvates, and will usually be used without derivatization.

For a review on suitable salts, see “Handbook of Pharmaceutical Salts: Properties, Selection, and Use” by Stahl and Wermuth (Wiley-VCH, Weinheim, Germany, 2002).

A pharmaceutically acceptable salt of a component suitable for use in the present invention may be readily prepared by mixing together solutions of a component compound and the desired acid or base, as appropriate. The salt may precipitate from solution and be collected by filtration or may be recovered by evaporation of the solvent. The degree of ionisation in the salt may vary from completely ionised to almost non-ionised.

The components suitable for use in the combinations of the present invention include the compounds as hereinbefore defined, polymorphs, prodrugs, and isomers thereof (including optical, geometric and tautomeric isomers).

Usually, components for use in the invention will be administered as a formulation in association with one or more pharmaceutically acceptable excipients. The choice of excipient will to a large extent depend on factors such as the particular mode of administration, the effect of the excipient on solubility and stability, and the nature of the dosage form. The components may be present in the same dosage form in accordance with the first aspect of the invention, or they may be present in separate dosage forms, for example as encompassed by the fourth aspect of the invention.

Pharmaceutical compositions suitable for delivering the compounds suitable for use in the combinations of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

Preferably, the compounds suitable for use in the combinations of the invention are administered orally, and therefore the formulations, uses, methods and products of the invention will be suitable for, or involve, oral administration. Oral administration may involve swallowing, so that the compound enters the gastrointestinal tract, or buccal or sublingual administration may be employed by which the compound enters the blood stream directly from the mouth. Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations. Tablets and capsules are preferred. Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet. The components suitable for use in the combinations of the invention may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 wt % to 25 wt %, preferably from 5 wt % to 20 wt % of the dosage form. Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate. Tablets may also optionally comprise surface active agents, such as sodium lauryl sulfate and polysorbate 80, and glidants such as silicon dioxide and talc. When present, surface active agents may comprise from 0.2 wt % to 5 wt % of the tablet, and glidants may comprise from 0.2 wt % to 1 wt % of the tablet. Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 wt % to 10 wt %, preferably from 0.5 wt % to 3 wt % of the tablet. Other possible ingredients include anti-oxidants, colourants, flavouring agents, preservatives and taste-masking agents. Exemplary tablets contain up to about 80% drug, from about 10 wt % to about 90 wt % binder, from about 0 wt % to about 85 wt % diluent, from about 2 wt % to about 10 wt % disintegrant, and from about 0.25 wt % to about 10 wt % lubricant. Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated. The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

Solid formulations for oral administration may be formulated to be immediate and/or modified release. Modified release formulations include delayed-, sustained-, pulsed-, controlled-, targeted and programmed release. Details of other suitable release technologies such as high energy dispersions and osmotic and coated particles are to be found in Verma et al, Pharmaceutical Technology On-line, 25(2), 1-14 (2001). The use of chewing gum to achieve controlled release is described in WO 00/35298.

The components suitable for use in the combinations of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques. Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilisation, may readily be accomplished using standard pharmaceutical techniques well known to those skilled in the art. The solubility of components used in the preparation of parenteral solutions may be increased by the use of appropriate formulation techniques, such as the incorporation of solubility-enhancing agents. Formulations for parenteral administration may be formulated to be immediate and/or modified release. Modified release formulations include delayed-, sustained-, pulsed-, controlled-, targeted and programmed release. Thus components for use in the invention may be formulated as a solid, semi-solid, or thixotropic liquid for administration as an implanted depot providing modified release of the active compound. Examples of such formulations include drug-coated stents and PGLA microspheres.

The components suitable for use in the combinations of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J. Pharm. Sci, 88 (10), 955-958 by Finnin and Morgan (October 1999). Other means of topical administration include delivery by electroporation, iontophoresis, phonophoresis, sonophoresis and microneedle or needle-free (e.g. Powderject®, Bioject®, etc.) injection. Formulations for topical administration may be formulated to be immediate and/or modified release. Modified release formulations include delayed-, sustained-, pulsed-, controlled-, targeted- and programmed-release.

The components suitable for use in the combinations of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin. The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid. Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or HPMC), blisters and cartridges for use in an inhaler or insufflator may be formulated to contain a powder mix of active ingredient(s), a suitable powder base such as lactose or starch and a performance modifier such as I-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose. A suitable solution formulation for use in an atomiser using electrohydrodynamics to produce a fine mist may contain from 1 μg to 20 mg of active ingredient(s) per actuation and the actuation volume may vary from 1 μl to 100 μl. A typical formulation may comprise active ingredient(s), propylene glycol, sterile water, ethanol and sodium chloride. Alternative solvents which may be used instead of propylene glycol include glycerol and polyethylene glycol. Suitable flavours, such as menthol and levomenthol, or sweeteners, such as saccharin or saccharin sodium, may be added to those formulations intended for inhaled/intranasal administration. Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

The components suitable for use in the combinations of the invention may be administered rectally or vaginally, for example, in the form of a suppository, pessary, or enema. Cocoa butter is a traditional suppository base, but various alternatives may be used as appropriate. Formulations for rectal/vaginal administration may be formulated to be immediate and/or modified release. Modified release formulations include delayed-, sustained-, pulsed-, controlled-, targeted- and programmed-release. The components suitable for use in the combinations of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration. Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

In accordance with the fourth aspect of the invention, two or more pharmaceutical formulations may conveniently be combined in the form of a kit suitable for co-administration of the compositions. Thus, the kit of the invention comprises two or more separate pharmaceutical formulations and means for separately retaining said formulations, such as a container, divided bottle, or divided foil packet. An example of such a kit is the familiar blister pack used for the packaging of tablets, capsules and the like. The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

For the avoidance of doubt, references herein to “treatment” include references to curative, palliative and prophylactic treatment.

Suitable dosages of the components for use in the combinations of the invention will depend on the component concerned, the condition to be treated and the weight of the patient. However, in general, a suitable daily dose of 5-alpha reductase inhibitor is in the range 0.01-10 mg, for example 0.5 to 5 mg for finasteride and 0.05 to 0.5 mg for dutasteride. In general, a suitable daily dose of PDE5 inhibitor is in the range 0.1-120 mg: for example 2.5-100 mg for sildenafil citrate; 0.5-200 mg for 5-[2-ethoxy-5-(4-ethyl-piperazine-1-sulphonyl)-pyridin-3-yl]-3-ethyl-2-[2-methoxy-ethyl]-2,6-dihydro-pyrazolo[4,3-d]pyrimidin-7-one, or a pharmaceutically acceptable salt thereof; and 0.5-20 mg for vardenafil, tadalafil, and pharmaceutically acceptable salts thereof.

Specific preferred combinations of the invention are:
sildenafil, or a pharmaceutically acceptable salt thereof+finasteride, or a pharmaceutically acceptable salt or solvate thereof;
sildenafil, or a pharmaceutically acceptable salt thereof+dutasteride, or a pharmaceutically acceptable salt or solvate thereof;
5-[2-ethoxy-5-(4-ethyl-piperazine-1-sulphonyl)-pyridin-3-yl]-3-ethyl-2-[2-methoxy-ethyl]-2,6-dihydro-pyrazolo[4,3-d]pyrimidin-7-one, or a pharmaceutically acceptable salt thereof+finasteride, or a pharmaceutically acceptable salt or solvate thereof; and
5-[2-ethoxy-5-(4-ethyl-piperazine-1-sulphonyl)-pyridin-3-yl]-3-ethyl-2-[2-methoxy-ethyl]-2,6-dihydro-pyrazolo[4,3-d]pyrimidin-7-one, or a pharmaceutically acceptable salt thereof+dutasteride, or a pharmaceutically acceptable salt or solvate thereof.

The typical weight ratio of active ingredients [PDE5 inhibitor:5-alpha reductase inhibitor] in these specific preferred combinations may vary from 1:10 to 10:1, for example 1:4 to 4:1, 1:3 to 3:1, 1:2 to 2:1, and includes 1:1.

### EXAMPLE 1

### Immediate Release Tablet Containing Finasteride and Sildenafil

Tablets having the following composition are prepared using conventional methods:

Core

| | | |
|---|---|---|
| Finasteride | 5.0 | mg |
| Sildenafil citrate | 25.0 | mg |
| cellulose, microcrystalline | 53.4 | mg |
| calcium hydrogen phosphate dihydrate | 18.0 | mg |
| sodium starch glycollate | 6.0 | mg |
| magnesium stearate | 0.4 | mg |
| colloidal anhydrous silica | 0.2 | mg |

Coating

| | | |
|---|---|---|
| Methylhydroxypropyl cellulose | 1.5 | mg |
| cellulose, microcrystalline | 0.3 | mg |
| stearic acid | 0.6 | mg |
| titanum dioxide E 171 | 0.6 | mg |

### EXAMPLE 2

### Immediate Release Tablet Containing Finasteride and 5-[2-ethoxy-5-(4-ethyl-piperazine-1-sulphonyl)-pyridin-3-yl]-3-ethyl-2-[2-methoxy-ethyl]-2,6-dihydro-pyrazolo[4,3-d]pyrimidin-7-one

Tablets having the following composition are prepared using conventional methods:

Core

| | | |
|---|---|---|
| Finasteride | 5.0 | mg |
| 5-[2-ethoxy-5-(4-ethyl-piperazine-1- | 5.0 | mg |
| sulphonyl)-pyridin-3-yl]-3-ethyl-2- | | |
| [2-methoxy-ethyl]-2,6-dihydro- | | |
| pyrazolo[4,3-d]pyrimidin-7-one | | |
| cellulose, microcrystalline | 53.4 | mg |
| calcium hydrogen phosphate dihydrate | 18.0 | mg |
| sodium starch glycollate | 6.0 | mg |
| magnesium stearate | 0.4 | mg |
| colloidal anhydrous silica | 0.2 | mg |

Coating

| | | |
|---|---|---|
| Methylhydroxypropyl cellulose | 1.5 | mg |
| cellulose, microcrystalline | 0.3 | mg |
| stearic acid | 0.6 | mg |
| titanum dioxide E 171 | 0.6 | mg |

### BIOLOGICAL EXAMPLES

Experimental models of BPH involving bladder outflow obstruction have been developed in a number of animal species. These models, which involve the placement of a ligature or disc around the urethra, mimic prostatic occlusion of the urethra and result in the appearance of non-voiding or unstable contractions of the bladder on cystometrical evaluation [Levin et al (2000) In: Prostatic Diseases (eds Lepor and Oesterling), WB Saunders & Co.]. In addition these models reproduce many of the LUT symptoms associated with multiple forms of OAB including increased voiding frequency and decreased functional voiding capacity.

The beneficial effects of the combinations of the invention may be demonstrated in the following mouse model of LUTS associated with BPH.

A mouse model of short term urethral obstruction has been characterised and demonstrated to show increased voiding frequency and the presence of non-voiding contractions, coupled with a reduced bladder capacity (Schroder et al. (2003) J. Urol. 170, 1017-1021). The advantage of this model is that it closely mimics the bladder dysfunction observed in BPH patients and LUTS associated with other overactive bladder conditions.

### Materials and Methods

Animals: DBA/1LacJ mice are used for the studies, available from Charles River Laboratories, UK. After arrival, the mice are housed for 6 weeks under identical conditions under a 12 hours light/dark photocycle, food and water are provided ad libitum.

The mice are randomly divided into 3 groups each. One third receives bladder outlet obstruction (BOO) as described below, one third receives sham surgery. The remaining mice serve as unoperated controls.

Surgical procedure: The mice in the BOO group are anaesthetized with ketamine (Ketalar®, Parke Davis, Barcelona, Spain; 100 mg/kg IP) and xylazine (Rompun®, Bayer, Leverkusen, Germany, 15 mg/kg IP). The obstruction is created by a standardized method as described in Schroder, et. al, 2003J. Urol. 170, 1017-1021. Sham operated animals receive surgery similarly, without tying the obstruction.

At day 5 after the obstruction a polyethylene catheter (PE, ID 0.38 mm, OD 0.61 mm) with a small cuff is inserted in the bladder dome and secured with a purse-string suture (7-0 silk). The obstructing ligature remains in place. The catheter is tunnelled subcutaneously, led out on the back of the neck, and surgically secured. Control animals receive the bladder catheter 2 days prior to cystometry.

Cystometry: Two days after insertion of the catheter (7 days after creation of the obstruction), the cystometric investigation is performed without any anaesthesia or restraint. The mice are placed into a metabolic cage (Gazzada, Buguggiatade, Italy). The bladder catheter is connected to a pressure transducer, which in turn is connected to a Grasse 7E Polygraph recorder. The bladder is continuously filled with saline at room temperature by means of a microinjecton pump (CMA 100, Carnegie Medicine, Solna, Sweden), at a filling speed of 25 μl/min.

The amount of voided urine is measured by means of a fluid collector, connected to a force displacement transducer (FT 03 D; Grass instrument Co., MA, USA). After a stabilization period of 60-80 minutes, in which the bladder is continuously filled, reproducible voiding patterns are achieved and recorded over a period of 30 minutes. The following parameters are measured: Micturition interval (time between 2 voids), baseline pressure (lowest pressure between 2 voids), threshold pressure (pressure immediately before micturition was initiated), micturition pressure (maximum voiding pressure), and micturition volume. Residual urine is emptied manually 3 times at the end of the cystometry and measured. Bladder capacity is calculated as the amount of saline infused into the bladder between 2 voids, plus the average amount of residual urine.

The animals are continuously observed in order to distinguish between moving artifacts and non-voiding bladder contractions. The surface of the collecting-funnel under the grid of the metabolic cage was sprayed with a thin layer of silicone.

1. A pharmaceutical formulation comprising a PDE5 inhibitor and a 5-alpha reductase inhibitor; and pharmaceutically acceptable salts and solvates thereof.

2. The formulation of claim 1, wherein the PDE5 inhibitor is selected from sildenafil; tadalafil; vardenafil; 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one; 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one; 5-[2-ethoxy-5-(4-ethyl-piperazine-1-sulphonyl)-pyridin-3-yl]-3-ethyl-2-[2-methoxy-ethyl]-2,6-dihydro-pyrazolo[4,3-d]pyrimidin-7-one; 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide (TA-1790); 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide (DA 8159); and pharmaceutically acceptable salts and solvates thereof.

3. The formulation of claim 1, wherein the PDE5 inhibitor is selected from sildenafil; tadalafil;
vardenafil; DA-8159; and 5-[2-ethoxy-5-(4-ethyl-piperazine-1-sulphonyl)-pyridin-3-yl]-3-ethyl-2-[2-methoxy-ethyl]-2,6-dihydro-pyrazolo[4,3-d]pyrimidin-7-one; and pharmaceutically acceptable salts and solvates thereof.

4. The formulation of claim 1, wherein the PDE5 inhibitor is selected from sildenafil; 5-[2-ethoxy-5-(4-ethyl-piperazine-1-sulphonyl)-pyridin-3-yl]-3-ethyl-2-[2-methoxy-ethyl]-2,6-dihydro-pyrazolo[4,3-d]pyrimidin-7-one; and pharmaceutically acceptable salts and solvates thereof.

5. The formulation of claim 1, wherein the 5-alpha reductase inhibitor is selected from finasteride; dutasteride; izonsteride; idronoxil; epristeride; serenoa repens; PHL 00801; and pharmaceutically acceptable salts and solvates thereof.

6. The formulation of claim 1, wherein the 5-alpha reductase inhibitor is selected from finasteride; dutasteride; and pharmaceutically acceptable salts and solvates thereof.

7. A method of treatment of LUTS, comprising simultaneous, separate or sequential administration of a PDE5 inhibitor and a 5-alpha reductase inhibitor, or a pharmaceutically acceptable salt or solvate thereof, to a patient in need of such treatment.

8. The method of claim 7, wherein the LUTS is urgency, frequency, nocturia, or urge incontinence.

9. The method of claim 7, wherein the PDE5 inhibitor is sildenafil, or a pharmaceutically acceptable salt thereof, and the 5-alpha reductase inhibitor is finasteride, or a pharmaceutically acceptable salt or solvate thereof.

10. The method of claim 7, wherein the PDE5 inhibitor is 5-[2-ethoxy-5-(4-ethyl-piperazine-1-sulphonyl)-pyridin-3-yl]-3-ethyl-2-[2-methoxy-ethyl]-2,6-dihydro-pyrazolo[4,3-d]pyrimidin-7-one, or a pharmaceutically acceptable salt thereof, and the 5-alpha reductase inhibitor is finasteride, or a pharmaceutically acceptable salt or solvate thereof.

11. The method of claim 7, wherein the PDE5 inhibitor is 5-[2-ethoxy-5-(4-ethyl-piperazine-1-sulphonyl)-pyridin-3-yl]-3-ethyl-2-[2-methoxy-ethyl]-2,6-dihydro-pyrazolo[4,3-d]pyrimidin-7-one, or a pharmaceutically acceptable salt thereof, and the 5-alpha reductase inhibitor is dutasteride, or a pharmaceutically acceptable salt or solvate thereof.

12. The formulation of claim 1, wherein the PDE5 inhibitor is sildenafil, or a pharmaceutically acceptable salt thereof, and the 5-alpha reductase inhibitor is dutasteride, or a pharmaceutically acceptable salt or solvate thereof.

13. The formulation of claim 1, wherein the PDE5 inhibitor is 5-[2-ethoxy-5-(4-ethyl-piperazine-1-sulphonyl)-pyridin-3-yl]-3-ethyl-2-[2-methoxy-ethyl]-2,6-dihydro-pyrazolo[4,3-d]pyrimidin-7-one, or a pharmaceutically acceptable salt thereof, and the 5-alpha reductase inhibitor is finasteride, or a pharmaceutically acceptable salt or solvate thereof.

14. The formulation of claim 1, wherein the PDE5 inhibitor is 5-[2-ethoxy-5-(4-ethyl-piperazine-1-sulphonyl)-pyridin-3-yl]-3-ethyl-2-[2-methoxy-ethyl]-2,6-dihydro-pyrazolo[4,3-d]pyrimidin-7-one, or a pharmaceutically acceptable salt thereof, and the 5-alpha reductase inhibitor is dutasteride, or a pharmaceutically acceptable salt or solvate thereof.
